(19) United States
(12) Patent Application Publication
(10) Pub. No.: US 2010/0291705 A1
(43) Pub. Date: Nov. 18, 2010

(54) ANTIGEN EXPOSING MICELLE AND UNORDERED AGGREGATE

(75) Inventors: Laura Varas, Malmo (SE); Anna Ketelsen, Alingsas (SE); Hakan Eriksson, Lund (SE)

Correspondence Address: MERCHANT & GOULD PC, P.O. BOX 2903, MINNEAPOLIS, MN, 55402-0903, US

(73) Assignee: EURO-DIAGNOSTICA AB, Malmo (SE)

(21) Appl. No.: 12/668,733

(22) PCT Filed: Jul. 10, 2008
(86) PCT No.: PCT/EP2008/059045

§ 371 Date: Jul. 2, 2010

Related U.S. Application Data
(60) Provisional application No. 60/959,293, filed on Jul. 12, 2007.

(30) Foreign Application Priority Data
Jul. 12, 2007 (SE) 0701692-6

(51) Int. Cl. G01N 33/566

(52) U.S. Cl. 436/501

(57) ABSTRACT

An antigen exposing micelle or unordered aggregate comprising at least one carrier, at least one epitope and at least one anchoring molecule, wherein said anchoring molecule comprises at least one anchoring part, intended to anchor the antigen exposing micelle to a surface.

### FIELD OF THE INVENTION

This invention relates to the field of antigens. More specifically it relates to an antigen exposing micelle or unordered aggregate, which comprises an anchoring part, manufacture of said micelle or unordered aggregate, a kit comprising said micelle or unordered aggregate, use of said micelle or unordered aggregate and/or said kit and a method to detect analytes by use of said micelle or unordered aggregate.

### BACKGROUND OF THE INVENTION

Auto-antibodies directed against hydrophobic antigens are common in several autoimmune diseases and disorders. For example, antibodies against gangliosides have been described in diverse neurodegenerative diseases, and antibodies against different phospholipids, or phospholipid associated proteins, are hallmarks of antiphospholipid syndrome, where these antibodies have been linked to an increased risk of thrombosis as well as recurrent fetal loss. Early, easy and reliable detection of such antibodies is desirable, both in diagnosis and in treatment. To enable early detection, the assay of choice must be sensitive and reproducible.

Auto-antibodies are commonly detected by standard immune-assays, such as enzyme linked immuno sorbent assay (ELISA). The method utilizes adsorption of proteins or other water-soluble molecules on polystyrene, and is mainly used for assays of water-soluble antigens or haptens.

Hydrophobic antigens are often un-soluble in water and may precipitate. Further, they may form aggregates in water. In the form of aggregates, the molecules are not adsorbed on surfaces, such as polystyrene, and therefore not well detected by antibodies in an ELISA. To circumvent these problems, hydrophobic molecules have been solubilized in organic solvents, such as ethanol, and dried onto multi-well plates. However, drying the hydrophobic molecules onto a surface easily results in multi-layer adsorption.

Furthermore the conformation of a molecule being an antigen might differ from its native conformation when being adsorbed to a surface. This non-native conformation might not be recognized by the antibody, as the specificity and affinity of antibodies to antigens are dependent on the conformation of the antigen.

During the different incubation and washing steps of an ELISA, the molecules deposited on a surface will, at least to some extent, be released into the surrounding media. This will affect the performance of the assay and introduce large inter- and intra-assay variations.

The washings steps in immuno assays, such as in an ELISA, are necessary to reduce the background resulting from unspecific binding of other molecules than the analytes. Preferably, detergents are used in such washings steps to reduce the background, but when analyzing hydrophobic antigens detergents should not be used according to state of the art (see for example WO 2007/002178 discussed below), as the hydrophobic antigens are thought to be washed away. Instead, buffers used in such washing steps may include a protein, such as BSA, as disclosed by Powers at al in Endocrinology 1984, 114:2 pp 1338-1343. Although proteins in such buffers will, at least to some extent, replace unspecifically bound material, there still is a demand for more effective washing steps when analysing hydrophobic antigens.

Furthermore, a procedure wherein hydrophobic antigens are adsorbed directly onto a surface, as described above, will not allow reliable detection of low amounts of auto-antibodies.

Amphiphilic molecules form various aggregates/structures in water and the nature of the amphiphilic molecules determines the form of the aggregate.

Ordered aggregates may for instance be spheres of amphiphilic molecules, i.e. micelles, layers of amphiphilic molecules facing each other forming a lamellar phase or structure, e.g. liposomes, a tubular arrangement called hexagonal phase or various cubic phases/structures.

Further, aggregates/structures formed by amphiphilic molecules in water may also be unordered in their form, i.e. nor exists as micelles neither as ordered double-layer(s), such as liposomes.

It is also believed that the lamellar phase can be in liquid crystalline phase and in a fluid phase and that the structural phase of the various forms of aggregates of amphiphilic molecules is influenced by the ratio of amphiphilic molecules present, temperature, hydration, pressure and ionic strength (and type). (J. M. Seddon, R. H. Templer. Polymorphism of Lipid-Water Systems, from the Handbook of Biological Physics, Vol. 1, ed. R. Lipowsky, and E. Sackmann. 1995, Elsevier Science B.V. ISBN0-444-81975-4.)

Ordered double-layers, such as liposomes, are labile structures, which easily are disrupted by detergents, such as detergents commonly used in the washing steps of immuno assays, e.g. ELISA. Accordingly, detergents can not be used in the washing steps, when liposomes are used in immuno assays. Consequently, molecules other than the analyte unspecifically adsorbed in a liposome based assay will not be removed during washing steps not employing detergents.

U.S. Pat. No. 5,776,487 relates to immunoassays utilizing novel liposome reagents having a ligand associated with or incorporated into the liposome to facilitate the detection of analyte in a patient sample

WO 2007/002178 relates to a method for immobilizing a lipoidal antigen, comprising cardiolipin, lecithin, and cholesterol, on a solid support, such as a nitrocellulose membrane. Further it relates to the diagnosis of syphilis.

Powers at al Endocrinology 1984, 114:2 pp 1338-1343 discloses a radioassay comprising immobilized, complex mixtures of several gangliosides for detecting antiganglioside antibodies.

Accordingly there is a current lack of assays to detect and/or quantify auto-antibodies against lipophilic antigens, which not suffers from the limitations discussed above.

### SUMMARY OF THE INVENTION

The present invention preferably seeks to mitigate, alleviate, circumvent or eliminate one or more of the above-identified deficiencies in the art and disadvantages singly or in any combination and solves at least the above mentioned problems by providing an antigen exposing micelle or unordered aggregate. Said antigen exposing micelle or unordered aggregate comprises at least one carrier, at least one epitope and at least one anchoring molecule, wherein said anchoring molecule comprises at least one anchoring part, intended to anchor the antigen exposing micelle or unordered aggregate to a surface.

In another aspect, there is provided a kit comprising antigen exposing micelle or unordered aggregate and least one device comprising at least one surface to which said anchoring part of said antigen exposing micelle or unordered aggregate has affinity.

In another aspect, there is provided a method to manufacture an antigen exposing micelle or unordered aggregate. Such a method comprises the steps of: dissolving at least one carrier, at least one anchoring molecule in a solvent, comprising a non-polar solvent; evaporating the solvent; dispersing the residue in an aqueous solvent; and sonicating the resulting mixture.

In another aspect, an antigen exposing micelle or unordered aggregate or a kit comprising antigen exposing micelle or unordered aggregate and least one device comprising at least one surface to which said anchoring part of said antigen exposing micelle or unordered aggregate has affinity, may be used to detect and/or quantify an analyte, such as an auto-antibody, which has affinity for said epitope.

In another aspect an analyte, such as an auto-antibody, which has affinity for said epitope, may be detected and/or quantified by: providing antigen exposing micelle or unordered aggregate; binding said micelle or unordered aggregate to a surface; exposing said bound micelle or unordered aggregate to a sample, which sample comprises an analyte, which analyte has affinity for said epitope; washing said bound micelle or unordered aggregate with a solution comprising a detergent; and detecting and/or quantifying said analyte.

Further aspects of the invention appear from the description and the appended claims.

### BRIEF DESCRIPTION OF THE DRAWINGS

These and other aspects, features and advantages of which the invention is capable of will be apparent from the following description of illustrative embodiments and examples of the present invention, reference being made to the accompanying drawings, in which

FIG. 1 depicts how the ratio between the carrier, e.g. lysophosphatidylcholine, and the antigen, e.g. GM-1, according to one embodiment of the invention, may affect the sensibility of the assay.

FIG. 2 depicts the anchoring effect of the anchoring part.

FIG. 3 depicts the titration of a rabbit anti GM-1 serum.

FIG. 4 depicts detection of antibodies in serum.

FIG. 5 depicts detection of an antigen associated to a phospholipid.

FIG. 6-9 depicts detection antibodies against and binding of antibodies to cardiolipin and to a complex between cardiolipin and β2-GPI.

### DETAILED DESCRIPTION OF EXAMPLE EMBODIMENTS

### Definitions

In the context of the present application and invention, the following definitions apply:

The term “carrier” is intended to mean a molecule, which comprises a lipophilic part and a hydrophilic part.

The term “anchoring molecule” is intended to mean a molecule, which comprise a lipophilic part and an anchoring part.

The term “anchoring part” is intended to mean a part, which has affinity for a specific group or a specific type of surface.

The terms “lipophilic” and “hydrophilic” are adopted from “IUPAC Compendium of Chemical Terminology—the Gold Book” (http://goldbook.iupac.org/index.html) and are intended to mean the character of interaction of a particular atomic group/groups with the medium. In this context the term “lipophilic” is intended to mean fat-preferring and water-rejecting and the term “hydrophilic” is intended to mean water-preferring and fat-rejecting.

The term “ganglioside” is intended to mean a ceramide and an oligosaccharide forming a glycosphingolipid.

The term “biotin analogues” is intended to mean molecules having the ability to bind to avidin or streptavidin and having essentially the same binding function to avidin or streptavidin as biotin, such as having a dissociation constant of ≦10⁻⁶.

The term “kit” is intended to mean a collection of items used to perform an assay.

The term “nucleotide sequence” is intended to mean a nucleotide sequence with single stranded nucleotides.

The term “derivative” is intended to mean a molecule similar to an original molecule, wherein at least one part present in the original molecule is lacking and/or wherein at least one part not found in the original is present in said derivative.

The term “biotinylated” is intended to mean that biotin, biotin analogues or other biotin molecules, having the ability to bind to avidin, streptavidin and derivatives thereof have been covalently bound to a molecule, optionally via a linker.

The term “coated surface” or similar wordings are intended to mean that said surface has been modified through non-covalent or covalent binding of molecules and/or atoms to said surface.

The term “auto-antibody” is intended to mean an antibody specific for a self-antigen.

The term “self-antigen” is intended to mean antigens of an organisms own cells and cell products.

The term “binding pair” is intended to mean a pair wherein the two members of the pair have affinity for each other.

Antigen Exposing Micelles and Unordered Aggregates

An embodiment according to the present invention relates to an antigen exposing micelle or unordered aggregate comprising at least one carrier, at least one epitope and at least one anchoring molecule, wherein said anchoring molecule comprises an anchoring part, intended to anchor the structure to a surface. Said carrier may contain said epitope, but is mainly intended to provide the micelle or unorderedaggregate with stability and the epitope is then part of a lipophilic antigen different from said carrier.

It was found possible to incorporate two or more different lipophilic structures, i.e. a lipophilic antigen comprising said epitope and an anchoring molecule, in the same micelle or unordered aggregate. Surprisingly, such micelles and unordered aggregate where found to not be disrupted by detergents. This finding is in contrast to common knowledge in the art.

The carrier may be of natural, synthetic or semi-synthetic origin or a mixture thereof. Similarly, also the part of the anchoring molecule not being the anchoring part, may be of natural, synthetic or semi-synthetic origin or a mixture thereof. Structures such as micelles are, by the one skilled in the art, known to be able to be formed in water by molecules that comprise both lipohilic part(s) and hydrophilic part(s), e.g. amphiphilic molecules.

Compared to fluid lamellar structures, such as liposomes, micelles and unordered aggregates have the advantage of being more stable structures.

As unordered aggregates are non-fluid structures they are very stable and resistant to solutions comprising detergents.

Further, micelles have a defined shape and structure. A micelle will expose an incorporated lipophilic antigen in its native configuration and is therefore a suitable structure to present epitopes of lipophilic antigens to a surrounding hydrophilic medium.

An unordered aggregate as disclosed herein, is to be distinguished from other types of lipophilic structures such as micelles and fluid lamellar structures, such as liposomes, which all are ordered structures. An unordered aggregate is, in contrast to fluid lamellar structures, to be regarded as a less fluid, such as a non-fluid or a solid, structure. It may be regarded as an amorphous type of structure, in contrast to fluid lamellar structures, which may be regarded as fluid. Furthermore, an unordered aggregate will not, in contrast to liposomes, enclose any solvent.

An unordered aggregate will also, at least to some extent, expose an incorporated lipophilic antigen in its native configuration and is therefore also a suitable structure to present epitopes of lipophilic antigens to a surrounding hydrophilic medium. Furthermore, due to the anchoring part such unordered aggregate as disclosed herein are easily bound to a surface as further disclosed herein.

It is also suitable, in terms of binding the antigen exposing structure to a surface, that the structure is relatively stable.

Furthermore a micelle is, as an unordered aggregate also is, once formed, a relatively stable structure, and no or very little exchange of members of the micelle or the unordered aggregate with the surrounding media take place. This feature is important, for example, when the antigen presenting micelle or unordered aggregate is used to detect analytes in serum containing lipophilic components. In contrast to liposomes comprising lipophilic antigens, where an exchange will take place between the liposome and lipid aggregates in the serum, no or very little exchange was surprisingly found to take place between a micelle or unordered aggregate comprising lipophilic antigens and lipid aggregates in the serum.

Similarly, less uniform structures, i.e. unordered aggregates, may be formed using amphiphilic molecules not forming micelles in water. Phospholipids may form lamellar or bilayer structures in water, but may also form unordered aggregates. Depending on nature of the lipids a fluid lamellar phase or a liquid crystalline phase may be formed. Furthermore, a non-fluid amorphous phase, i.e. unordered aggregate, may be formed. As one example, unordered aggregates may be formed by mixing biotinylated phosphatidylethanolamine and cardiolipin. As disclosed herein unordered aggregate will have similarly advantages as micelles.

Accordingly another embodiment according to the present invention relates to an antigen exposing unordered aggregate, comprising at least one carrier, at least one epitope and at least one anchoring molecule, wherein said anchoring molecule comprises an anchoring part, intended to anchor the aggregate to a surface. Said carrier may contain said epitope, but is mainly intended to provide the aggregate with stability and the epitope is then part of a lipophilic antigen different from said carrier.

Another embodiment according to the present invention relates to an antigen exposing micelle, comprising at least one carrier, at least one epitope and at least one anchoring molecule, wherein said anchoring molecule comprises an anchoring part, intended to anchor the aggregate to a surface. Said carrier may contain said epitope, but is mainly intended to provide the aggregate with stability and the epitope is then part of a lipophilic antigen different from said carrier.

In contrast to a liposome comprising a lipophilic antigen, where the lipophilic antigen may present its epitope both to the surrounding medium and to the interior of the liposome, a micelle comprising a lipophilic antigen will always present the epitope to the surrounding medium. Consequently all antigens added to the micelle will present their epitopes to the surrounding media.

Micelles and unordered aggregates, such as the once disclosed herein was, in contrast to other types of structures, such as liposomes, fluid lamellar structures etc., which easily are disrupted, surprisingly found to withstand standard washing conditions used in immuno assays, e.g. ELISA. Said washing conditions do normally comprise a detergent, such as washing with an aqueous solution comprising Tween, such as 0.05% Tween.

The advantages discussed above and other advantages will be further explained below.

The size of the micelle might differ and depends upon which amphiphilic molecules used. In one embodiment the micelle has a diameter of about 5 nm to about 300 nm. I another embodiment the micelle has a diameter of about 5 nm to 100 nm.

Similarly, the size of unordered aggregates might differ and depends upon which amphiphilic molecules used. According to one embodiment, the unordered aggregate has such a small diameter that an aqueous solution comprising such aggregates appears clear. An aqueous solution comprising aggregates with a diameter exceeding 1 μm will appear milky. Accordingly, another embodiment relates to an unordered aggregate having a diameter of less than 1 μm, such as less than 500 nm or even less than 250 nm.

The carrier might be selected from one or several types of molecules selected from the group comprising fatty acids, such as stearic acid, behenic acid, linoleic acid, arachidonic acid, sphingolipids, such as lysosphingolipids, phospholipids, such as lysophospholipids, glycolipids, such as cerebrosides and gangliosides, such as GM-1, asialo-GM-1, GM-2, asialo-GM-2 and GM-3, steroids, such as cholesterol and phytosterols, and surfactants such as detergents.

In one embodiment the carrier forming the structure is chosen from molecules that are known to form micelles in concentrations above one micromolar (μM). Such molecules may be found among both natural and synthetic lysophospholipids.

In another embodiment more than one type of molecules are used as carrier. By using more than one type of carrier the properties of the antigen presenting micelle or unordered aggregate may be adjusted, which might be advantageous. Cholesterol is one example of a carrier, which may be added to form more stable micelles or unordered aggregates.

In another embodiment the micelle or unordered aggregate comprises natural or synthetic lysophosphatidylcholine. The fatty acid in lysophosphatidylcholine may be a saturated, mono-unsaturated or un-saturated fatty acid. One example of a lysophosphatidylcholine to be used as carrier is L-a-lysophosphatidylcholine from hen egg.

The epitope may be a part of a lipophilic antigen, which is poorly water-soluble. By incorporating an antigen in the antigen exposing micelle or unordered aggregate described herein, its apparent solubility in water may be increased and precipitation of lipophilic antigens may thereby be avoided. Accordingly, one embodiment relates to a micelle or unordered aggregate, wherein the epitope is part of a lipophilic antigen, which not is the carrier or the anchoring molecule.

One embodiment of the present invention relates to the use of a carrier, as disclosed herein, and an anchoring molecule, as disclosed herein, to incorporate a lipid antigen in a micelle or an unordered aggregate. While the lipid antigen is distinct from the carrier and the anchoring molecule, the carrier and the anchoring molecule may be the same or may be different. Further the present invention relates to the use of such a micelle or unordered aggregate with an incorporated a lipid antigen to immobilize the lipid antigen, such as binding it to a surface. Such an immobilized lipid antigen may be used to detect and/or quantify an analyte as further discussed herein. Due to the use of washing solutions containing detergent, the detection limit of the antigen may be reduced.

The epitope is typically situated in a hydrophilic part of the lipophilic antigen. By incorporating the lipophilic antigen in a micelle or unordered aggregate, such as herein described, the antigen might present the epitope to the surrounding medium in its native conformation. Thereby antibodies or other molecules with affinity for the antigen and present in the surrounding medium might recognize and bind to the antigen.

In one embodiment the epitope is part of the carrier, which forms the antigen presenting micelle or unordered aggregate. Without limitation one example of molecules that might act both as carrier and lipophilic antigen is gangliosides, such as GM-1, and cardiolipin. But, as discussed above, the carrier is preferably different from the antigen. The antigen presenting micelle then comprises at least three different components: a carrier, an anchoring molecule and a lipophilic antigen. In such an embodiment the carrier serves to form a stable micelle or unordered aggregate in which the anchoring molecule and the lipophilic antigen may be incorporated.

Further, also the anchoring molecule may serve to form the micelle or the unordered aggregate. If the anchoring serves to form the micelle, when a second carrier, if present, may serve to stabilize the micelle.

According to one embodiment antigen comprising the epitope may be selected from hydrophilic antigens known to form micelles in water, such as asialoganglioside GM-1, disialoganglioside GD-1a, disialoganglioside GD-1b and disialoganglioside GD-2. If a hydrophilic antigen known to form micelles in water is used, it may be preferably to include a carrier different from the antigen in the micelle or the aggregate as it may increase the sensitivity of an immuno assay employing the antigen exposing micelle or unordered aggregate. As shown in example 4, it was, for an example, found advantageous to include a carrier, such as lyso-phosphatidylcholine, in micelles comprising GM-1.

According to another embodiment the epitope may be selected from lipophilic antigens known to form unordered aggregates, such as cardiolipin and DNP-phosphatidylethanolamine. If a hydrophilic antigen known to form unordered aggregates in water is used, it may be less advantageous to include a carrier different from the antigen in the micelle or the aggregate.

The epitope might be selected from hydrophilic antigens to which antibodies formed in auto-immune diseases or disorders are directed. Without being limited to, examples of such antigens are gangliosides, such as GM-1, asialo-GM-1, GM-2, asialo-GM-2 and GM-3, cardiolipin, phospholipids, sphingolipids and derivatives thereof.

In another embodiment the epitope is part of a hapten such as DNP (dinitrophenyl). Said hapten is coupled to a molecule comprising a lipophilic part, such as phosphatidylethanolamine, to enable the incorporation in the micelle or the unordered aggregate, which will expose the hapten. In this way the present drawbacks of using haptens as antigens in immunoassays are eliminated. These drawbacks are similar to the one discussed above for lipophilic antigens.

The anchoring part, intended to anchor the antigen exposing micelle or unordered aggregate to a surface may be selected from members of specific binding pairs, such as biotin, biotin analogues such as norbiotin, homobiotin, oxybiotin, iminobiotin, desthiobiotin, diaminobiotin, biotin sulfoxide, biotin sulfone or other biotin molecules having the ability to bind to avidin, streptavidin and derivatives thereof, avidin, streptavidin, thiols, antigens, antibodies, haptens, nucleotide sequences, and derivatives or parts thereof. Other examples of members of specific binding pairs may also be used.

In one embodiment of the invention the anchoring part, intended to anchor the antigen exposing micelle or unordered aggregate to a surface may be selected biotin, biotin analogues such as norbiotin, homobiotin, oxybiotin, iminobiotin, desthiobiotin, diaminobiotin, biotin sulfoxide, biotin sulfone or other biotin molecules having the ability to bind to avidin, streptavidin and derivatives thereof.

By using a member of a specific binding pair, as anchoring part, it may be possible to anchor the micelle or unordered aggregate to a surface, especially if the surface is, at least partly, coated with the corresponding member of the binding pair. In contrast to an unspecific interaction, such as a hydrophobic interaction, e.g. between a lipid antigen and a polymeric surface, the anchoring of the micelle or unordered aggregate via a specific binding pair will make the anchoring less susceptible to the washing conditions used in immuno assays. By use of an anchoring part the lipophilic antigen present in the antigen presenting micelle or unordered aggregate bound to the surface by the anchoring part will not be easily washed away. Thereby unbound material present in the sample may be washed away.

Furthermore, as discussed above, detergents may, in contrast to common knowledge within the state of art (see for an example WO 2007/002178), be used to make this washing more effective and also material, which unspecifically has bound to the solid phase surface, may be washed away. Such washing will increase the sensitivity and lower the detection limit when the antigen presenting structure is used in immunoassays, e.g. ELISA.

In one embodiment the detergent used in such washings is tween-20. In another embodiment other detergents and concentrations used in immuno-assays when assaying hydrophilic antigens are used. Such detergents comprise triton X, tween-40 and tween-80. Such concentrations may be 0.1 wt % or less, 0.05 wt % or less or 0.01 wt % or less.

The use of biotin, biotin analogues such as norbiotin, homobiotin, oxybiotin, iminobiotin, desthiobiotin, diaminobiotin, biotin sulfoxide, biotin sulfone or other biotin molecules having the ability to bind to avidin, streptavidin and derivatives thereof as anchoring part, may due to their high affinities provide the bound micelle or unordered aggregate with increased resistance to detergents compared to other types members of specific binding pairs.

In another embodiment the anchoring part is biotin. Biotin is a well-known member of a specific binding pair. The corresponding member may be avidin or streptavidin. As biotin often has been used in specific binding pairs, the one skilled in the art is familiar to the use of biotin conjugates and how to conjugate biotin to other molecules.

Biotin conjugates of amphiphilic molecules are commercially available (Avanti polar lipids (Alabaster, Ala.) and activated biotin, which is simple to couple to other molecules, is commercially available (BioRad, Richmond, Pa.).

In another embodiment the anchoring part is conjugated to an amphiphilic molecule forming unordered structures in aqueous solution, i.e. a molecule not forming micelles or fluid lamellar structures to any large extent. One example of such amphiphilic molecule is phosphatidylethanolamine. Other examples are phosphatidyl serine or an amide or ester thereof and phosphatidic acid (1,2-Diacylglycerol 3-phosphate). Preferably should the anchoring part be covalently linked to an amphiphilic molecule not spontaneously forming micelles or fluid lamellar layers by it self, without addition of other molecules, in aqueous solution below 30° C.

In another embodiment the anchoring molecule comprises a phosphatidyl moiety, such as, but not limited to, phosphatidylethanolamine. In another embodiment anchoring part is conjugated to phosphatidylethanolamine.

In another embodiment the lipid part of the anchoring molecule comprises a functional group such as a hydroxyl, amino or carboxyl functionality to which an anchoring part easily may be coupled, as well known to the one skilled on the art.

In another embodiment the anchoring part is conjugated to an amphiphilic molecule comprising a glycerol moiety, which is di-substituted with fatty acids. Such fatty acids may be the same or the may differ. The may be saturated, mono-unsaturated or un-saturated fatty acids. One example of such a fatty acid is hexadecane acid.

In another embodiment the anchoring molecule is biotinylated phosphatidylethanolamine, which is commercially available (Invitrogen, Carlsbad, Calif.)

In yet another embodiment the anchoring molecule is GM-1. The corresponding binding member to GM-1 may then be cholera toxin.

In yet another embodiment the anchoring part is a hapten conjugated to a lipophilic molecule. The corresponding binding member to the hapten may then be an antibody.

Although haptens and epitopes of antigens may be used as anchoring parts, it may, according to one embodiment of the inventions and as disclosed herein, be advantageous to use biotin or biotin-analogues as anchoring part.

In yet another embodiment the antigen exposing micelle or unordered aggregate, is a micelle comprising at least one carrier selected from lysophosphatidylcholine, gangliosides, such as GM-1, a lipophilic antigen such as a ganglioside, e.g. GM-1, GM-2, asilio GM-1 and a biotinylated anchoring molecule, such as biotinylated phosphatidylethanolamine.

In yet another embodiment the antigen exposing micelle or unordered aggregate, is an unordered aggregate comprising a lipophilic antigen such as a cardiolipin or an antigen, wherein the epitope is part of a hapten and in which the hydrophobic part is a phospholipid, such as dinitrophenyl-phosphatidylethanolamine (DNP-PE) and a biotinylated anchoring molecule, such as biotinylated phosphatidylethanolamine.

In yet another embodiment the antigen exposing micelle or unordered aggregate comprises from about 1 to about 95 weight % of a carrier and from about 1 to about 50 weight % of a anchoring molecule.

In yet another embodiment the antigen exposing micelle or unordered aggregate comprises from about 1 to about 95 weight % of a carrier, from about 1 to about 50 weight % of a anchoring molecule and from about 1 to about 80% of a lipophilic antigen different from said carrier and said anchoring molecule.

In yet another embodiment the antigen exposing micelle or unordered aggregate comprises from about 20 to about 80 weight % of a carrier, from about 5 to about 20 weight % of a anchoring molecule and from about 10 to about 70% of a lipophilic antigen different from said carrier and said anchoring molecule.

In yet another embodiment the antigen exposing micelle or unordered aggregate comprises from about 20 to about 80 weight % of a first carrier, from about 5 to about 20 weight % of a second carrier, from about 5 to about 20 weight % of a anchoring molecule and from about 20 to about 70% of a lipophilic antigen different from said first and second carrier and said anchoring molecule. In such an embodiment the second carrier may be used to increase the incorporation of the lipophilic antigen in the antigen presenting micelle or unordered aggregate, to increase the stability of the antigen presenting micelle or unordered aggregate or to stabilize the structure of the antigen.

In yet another embodiment the antigen exposing micelle or unordered aggregate comprises from about 5 to about 20 weight % of a anchoring molecule. By limiting the amount of the anchoring molecule, a more efficient presenting of the antigen may be achieved.

Furthermore, in embodiments relating to unordered aggregates only comprising two types of components, i.e. an anchoring molecule and a lipophilic antigen, the amount of the anchoring molecule may be from about 5 to about 20 weight %. By limiting the amount of the anchoring molecule to such an amount, the presence of liposomes and other types of fluid lamellar layers in the solution comprising the unordered aggregate may be minimized.

A Kit Comprising an Antigen Exposing Micelle

A kit may comprise an antigen exposing micelle or unordered aggregate as described herein and a device comprising at least one surface to which the anchoring part of the antigen exposing micelle or unordered aggregate has affinity.

In one embodiment, a kit comprising an antigen exposing micelle or unordered aggregate, as describe herein, and a device comprising at least one surface is provided. The device may be selected from multi-well plates, such as 96-, 384- or 1536-well plates, test tubes, MALDI-TOF plates, paper strips, glass slides, beads, particles or any other surface used in immuno assays as well in array performance.

The surface of the device should have such properties that the anchoring part of the antigen exposing micelle or unordered aggregate has affinity for it. One way among others to provide a surface with such a property is to coat it. Members of different types of specific binding pairs can be used to coat the surface of the device and thereby enable anchoring of the antigen exposing micelle. Thereby different anchoring parts could be used depending on the specific demands in a specific case. If such a coated surface is exposed to a preparation comprising an antigen exposing micelle or unordered aggregate, as describe herein, the micelle or unordered aggregate may bind to the surface and the solution could be removed without removing the antigen exposing micelle. Subsequently, other solutions could be applied to the kit with the bound antigen exposing micelle. These solutions could comprise molecules, such as antibodies, specific for the exposed epitope. Thereby these molecules may be bound to the surface of the antigen exposing system, and any unbound material may be washed way without affecting the bound antigen exposing system or the molecule bound to it.

In another embodiment the coating comprises avidin or streptavidin. Avidin or streptavidin are well-known members of specific binding pairs. The corresponding member may be biotin, biotin analogues such as norbiotin, homobiotin, oxybiotin, iminobiotin, desthiobiotin, diaminobiotin, biotin sulfoxide, biotin sulfone or other biotin molecules having the ability to bind to avidin, streptavidin and derivatives thereof. As avidin or streptavidin often have been used in specific binding pairs, the one skilled in the art is familiar with how to coat avidin or streptavidin on surfaces, such as polystyrene plates.

In another embodiment the coating comprises biotin or a protein or a polymer covalently linked with biotin, biotin analogues such as norbiotin, homobiotin, oxybiotin, iminobiotin, desthiobiotin, diaminobiotin, biotin sulfoxide, biotin sulfone or other biotin molecules having the ability to bind to avidin, streptavidin and derivatives thereof. Other examples of members of specific binding pairs, such as antigens, haptens, antibodies, nucleotide sequences, and derivatives or parts thereof may also be used. As mentioned above, there are certain advantages by using such a member of a binding pair.

In another embodiment the coating comprises cholera toxin.

In another embodiment multiwell plates, such as non-porous plates, e.g. polystyrenplates, are used as surface to which the antigen exposing micelles or unordered aggregates are bound. A non-porous plate wherein the micelles or unordered aggregates may be bound to the surface rather than absorbed, have the advantage of being easy to wash. Accordingly, the washing steps in an immuno-assay, as disclosed herein, may be even more effective if a non-porous device is employed.

In another embodiment beads or particles are used to provide a surface to which the antigen exposing micelles and aggregates are bound.

In another embodiment the kit comprises the antigen exposing micelle or unordered aggregate bound to a surface or coating of a device. The surface or coating may be of the types discussed above. Such a kit is ready to use directly, without any additional steps, for the detection and quantification of analytes, e.g. of antibodies in serum, which has affinity for the exposed antigen.

Such a kit as have been describe above may further comprise a detection reagent, such as an antibody. Said detection reagent may have affinity for an analyte, such as antibody, which antibody may be an auto-antibody, wherein said analyte has affinity for the antigen exposed by the antigen presenting structure. The detection antibody may be an anti-human antibody with affinity for human antibodies. Further the detection antibody might be an antibody with affinity for antibodies from the species to which antibodies with affinity towards the lipophilic antigen belongs.

In another embodiment the detection antibody is selected from group consisting of antibodies against h-Ig, h-IgM, hIgG, h-IgA and h-IgE.

In another embodiment the detection antibody comprises a moiety to enable detection of the analyte to which the detection antibody may be bound. This means that the analyte bound to the antigen exposing micelle or unordered aggregate may be detected and the amount of it present quantified. As any unbound material in the sample, which comprises the analyte, as well as material unspecifically bound to the solid surface may be washed away as described above, any noise, which would negatively affect the detection and/or quantification, is reduced.

In another embodiment this moiety, which enables detection of bound antibodies, may be selected from the group consisting of florescent groups, such as FITC, radioactive groups, such as ¹²⁵I, enzymes, such as horseradish peroxidise or phosphatase, biotin, avidin. Use of such groups mean that the amount of bound analyte may automatically be detected and/or quantified, with e.g. a plate reader, as is well-known for a person skilled in the art.

In another embodiment the detection reagent may be protein A or protein G.

Method to Manufacture an Antigen Exposing Micelle or Unordered Aggregate.

An antigen exposing micelle or unordered aggregate might be manufactured by:
a) dissolving a carrier, an anchoring molecule and a lipophilic antigen, optionally the same as the carrier, in a solvent comprising a non-polar solvent, such as chloroform;
b) evaporating the solvent; and
c) dispersing the residue in an aqueous solvent, such as phosphate buffered saline (PBS), and sonicating the resulting mixture.

Examples of carriers, lipids comprising epitopes and anchoring molecules are described in the previous sections. As ratios of the parts to form the structures are.

In one embodiment, the resulting mixture is sonicated at or above the melting point of the components used. In such an embodiment, the resulting mixture may be sonicated at or above 50° C. In another embodiment, wherein the resulting mixture is sonicated at elevated temperature, the resulting apparently clear solution is allowed to cool down to room temperature, such as about 21° C., before use of the resulting structures. In another embodiment, wherein the resulting mixture is sonicated at elevated temperature, the resulting apparently clear solution is allowed to cool down to about 4° C., before use of the resulting structures.

Even though micelles or unordered aggregates may be the major type of structure present in a such a mixture as disclosed above, some of the components may also exists as ordered structures, such as liposomes and other types of fluid lamellar layers.

As the anchoring molecule will disturb ordered structures, such as liposomes and other types of fluid lamellar layers, the anchoring molecule will be enriched in micelles or unordered aggregates. Furthermore, structures not comprising an anchoring molecule as well as ordered structures, such as liposomes and other types of fluid lamellar layers, may, in contrast to micelles and unordered aggregates comprising an anchoring molecule, be washed away with an aqueous solution comprising a detergent. Accordingly, bound micelles and unordered aggregates comprising an anchoring molecule may be enriched.

Method to Manufacture a Kit

A kit comprising the antigen exposing micelle or unordered aggregate may be manufactured by exposing a device, which is coated with one of members of the binding pairs used to anchor the antigen exposing micelle or unordered aggregate, examples of which have been given above, to a solution comprising the antigen exposing micelle or unordered aggregate. Said kit may then be encapsulated. Such encapsulation means that the kit will be easier to transport and will also provide the kit with longer shelf life. Further, encapsulation does minimize the risk of contamination prior to use of the kit.

In another embodiment the kit may be manufactured by exposing a device, which is coated with one of members of the binding pairs used to anchor the antigen exposing micelle or unordered aggregate, examples of which have been given above, to an apparent solution comprising said antigen exposing micelle or unordered aggregate. Unbound material, which may include micelles or unordered aggregates not having any anchoring molecule incorporated within the structure, may then optionally be washed away. Such a washing step will remove structures and molecules, which not have bound properly to the surface and whereby could interfere in subsequent use of the kit. As the micelles and aggregates are relatively stable structures, detergents, such as Tween, may be used to make this washing step more effective. Then a second solution, which comprises water, carbohydrates, such as mannitol, dextran, and lactose or any other stabilizing molecule, may be added. Such a solution may provide the antigen presenting structures present in the kit with stability and thereby provide the kit with longer shelf life. Prior to encapsulation the device containing adsorbed antigen exposing micelles or unordered aggregates may be dried, such as lyophilized (freeze dried), to give an essentially dry device.

In contrast to other structures, such as fluid lamellar structures, e.g. liposomes, micelles and unordered aggregates are stable enough to withstand the refrigeration conditions used during freeze-drying.

A dry device may be easier to handle and transport. Furthermore it may be more robust than a device comprising a liquid medium. It may also have a longer shelf life.

Finally the kit is encapsulated and supplied as parts, one containing the device with adsorbed antigen exposuring structures and one part containing the detection reagent(s). As mentioned above, encapsulation may make the kit easier to transport and may also provide the kit with longer shell life. Further, encapsulation does minimize the risk of contamination prior to use of the kit.

Detection and/or Quantification of an Analyte by Use of an Antigen Presenting Structure or a Kit Comprising One

An antigen presenting micelle or unordered aggregate or a kit comprising such a micelle or unordered aggregate, and which has been described above, may be used to detect and/or quantify an analyte, which has affinity for the presented epitope, present in complex samples, such as plasma or serum, from a mammal.

In one embodiment the sample is mammal serum, such as a human serum from a patient possibly comprising auto-antibodies. By use of the antigen presenting micelle or unordered aggregate or a kit described herein the presence of auto-antibodies can be detected and eventually used to diagnose an autoimmune disease or disorder. Examples of diseases and disorders that may be diagnosed this way are peripheral neurophaties, such as Guillian Barre syndrome, antiphospholipid syndrome and artherosclerosis.

Due to the sensitivity, which partly is due to the stability of the micelles and aggregates, which renders the use of detergents in washing steps possible, of the method of detection described herein the diagnosis of auto-immune diseases at an early stage become possible. Early detection is important to be able to start the treatment of the disease or disorder as early as possible to avoid and minimize organ damage.

Furthermore, the detection of some lipophilic antigens is not possible by methods in the current state of the art. Extensive sample treatment before the detection and/quantification has been employed in attempts to decrease the complexity of samples, such as serum, in order to reduce the need for washing steps. Such pre-treatments of samples will give rise to a more time consuming assay, commonly associated with a reduced precision and accuracy. These shortcomings are solved by the present invention.

In another embodiments the present invention relates to a method of detecting and/or quantifying an analyte, such as an auto-antibody. The presence of auto-antibodies in a sample, such as serum, from a patient, may indicate that the patient suffers from an autoimmune disease or disorder. Such a method comprising the steps of; providing a micelle or unordered aggregate of the kind as herein described; binding said micelle or unordered aggregate to a surface; optionally washing away any unbound micelles or unordered aggregate, optionally by using a solution comprising a detergent, such as an aqueous solution comprising 0.05% Tween; exposing said bound micelle to a sample, which sample comprises an analyte, which analyte has affinity for said epitope; optionally washing away parts and components of the sample not bound to said epitope and parts and components unspecifically bound to said epitope by using a solution comprising a detergent, such as an aqueous solution comprising 0.05% Tween; and detecting and/or quantifying said analyte.

In another embodiments the present invention relates to a method of detecting and/or quantifying an analyte, such as an auto-antibody, such a method comprises at least one washing step, wherein a detergent is used.

Although the micelle or unordered aggregate normally would be bound to the surface before being exposed to the analyte, it is also possible to first expose the micelle or unordered aggregate to the analyte and then bind the micelle or unordered aggregate to the surface.

As disclosed herein, WO 2007/002178 relates to a method for immobilizing a lipoidal antigen, comprising cardiolipin, lecithin, and cholesterol, on a solid support, such as a nitrocellulose membrane. Further it relates to the diagnosis of syphilis. In the method of WO 2007/002178 it is disclosed that, similarly to current understandings, any washing step in such method as disclosed in WO 2007/002178 should be free from detergents.

In contrast to these well-established teachings, it was surprisingly found possible and indeed advantageous to include detergents in at least one of washing steps in such a method as disclosed herein.

Although the present invention has been described above with reference to specific illustrative embodiments, it is not intended to be limited to the specific form set forth herein. Other embodiments are possible within the scope of the appended claims.

### EXAMPLES

The examples given below are only intended to further illustrate the invention and are by no means intended to limit the scope of the invention as defined by the appended claims.

Material

Lysophosphatidylcholine (L-a-lysophosphatidylcholine from hen egg), gangliosides; monosialo ganglioside GM-1, asialoganglioside GM-1, disialoganglioside GD1a, and disialoganglioside GD1b (bovine brain), were from Sigma-Aldrich (St Louis, Mo.). Biotinylated phosphohoetanolamine (N-((6-(biotinyl)amino)hexanoyl)-1,2-dihexadecanoyl-sn-glycero-3-phosphoethanolamine (biotin-PE) and cardiolipin were from Invitrogen (Carlsbad, Calif.).

### Example 1

### Generation of Antigen Presenting Micelles Prepared by Mainly Using Micell Forming Reagents

Carriers, such as lysophospholipids, anchoring molecules, such as biotin-PE, and lipophilic antigens such as gangliosides e.g. GM-1, DNP-phosphatidylethanolamine mixed in different molar ratios, were dissolved in chloroform or a mixture of chloroform:methanol 1:1. The resulting mixture was then dried under nitrogen. The residue was dispersed in phosphate buffered saline (PBS) to a final concentration of 200 μg/ml, and sonicated at 50° C. for 10 min, until the solution appeared clear. The preparation with antigen presenting micelles was stored at 4° C. Prior to use it was sonicated again at 50° C. for 10 min before it was added to ELISA plates.

### Example 2

### Generation of Antigen Presenting Aggregates Prepared Reagents Spontaneously not Forming Fluid Lamellar Bilayers

Anchoring molecules, such as biotin-PE, and lipophilic antigens, such as cardiolipin or DNP-phosphatidylethanolamine, mixed in different molar ratios, were dissolved in chloroform. The resulting mixture was then dried under nitrogen. The residue was dispersed in phosphate buffered saline (PBS) to a final concentration of 200 μg/ml, and sonicated at 50° C. for 10 min or until the solution appeared clear. The preparation with antigen presenting aggregates was stored at 4° C. Prior to use it was sonicated again at 50° C. for 10 min before it was added to ELISA plates.

### Example 3

### Coating of ELISA Plates

96-well ELISA plates (NUNCmaxisorp) were coated with 100 ng streptavidin (Sigma-Aldrich) in PBS per well at 4° C. overnight or 2 h at 37° C. Plates were then blocked with 2% BSA (Cohn fraction V Sigma-Aldrich) or 0.5% gelatine (Sigma-Aldrich) in PBS for 1 h at room temperature.

### Example 4

### Importance of the Carrier

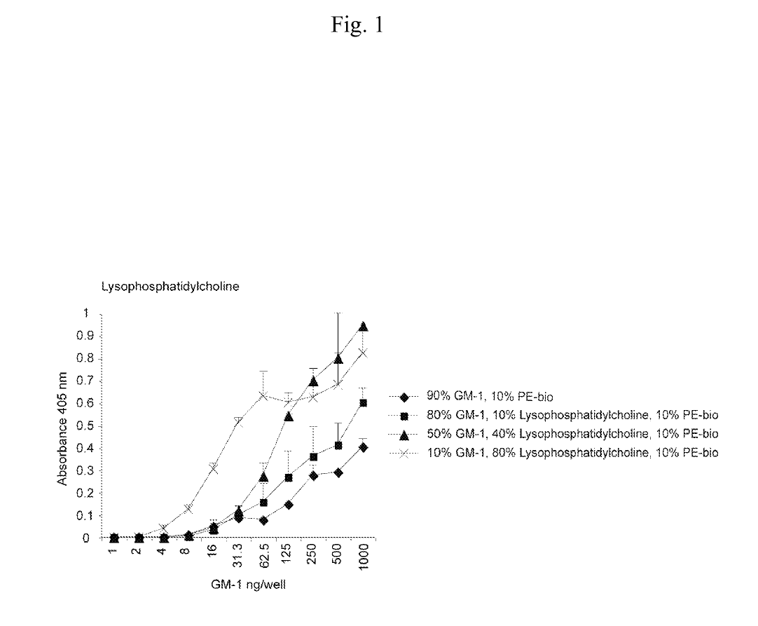

Preparations with antigen presenting micelles, prepared according to example 1, wherein the ratios between carrier (lyso-phosphatidylcholine), lipophilic antigen (GM-1) and anchoring molecule (biotin-PE) varied, were added at different concentrations (see FIG. 1), and the plate incubated for 1 h at room temperature.

To detect GM-1 in the different antigen presenting micelles, the plate was incubated with cholera toxin subunit B conjugated with horseradish peroxidase (CTB-HRP, Invitrogen), 2 μg/ml in PBS, for 1 h at RT. The plate was developed with tetramethylbenzidine (TMB substrate reagent set, BD Biosciences, San Diego, Calif.), and absorbance at 405 nm measured using an ELISA plate reader (PowerWave WS, Bio-Tek Instruments Inc., Winooski, Vt.) after 10-30 minutes. Between each incubation step the plate was washed three times with 0.05% Tween-20 in PBS, if not otherwise stated in figure.

As seen in FIG. 1 the incorporation of a carrier lipid (lysophosphatidylcholine FIG. 1) different from the lipophilic antigen, may lower the detection limit of the lipophilic antigen, i.e. GM-1.

### Example 5

### Anchoring Effect of Biotin-PE

Antigen presenting micelles containing GM-1 were prepared according to example 1 from GM-1, lyso-phosphatidylcholine, biotin-PE and incubated in ELISA plates with or without streptavidin coating. After incubation the plates were washed with PBS containing 0.05% Tween 20 and bound GM-1 was detected by HRP-labelled cholera toxin.

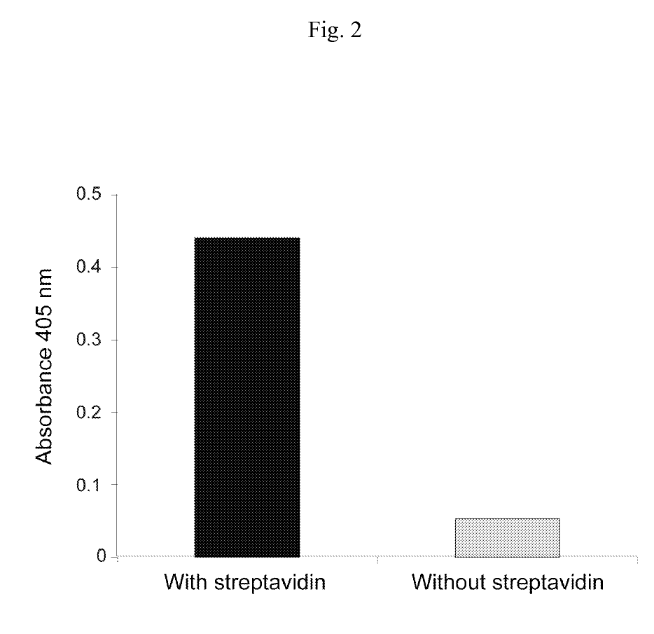

As seen from FIG. 2, nearly all antigen presenting micelles were washed away in the absence of streptavidin.

### Example 6

### Titration of Rabit-Anti-GM1 Serum

Antigen presenting micelles containing GM-1 was prepared according to example 1 from GM-1, lyso-phosphatidylcholine, biotin-PE and bound to ELISA plates coated with streptavidin. GM-1 was detected with rabbit anti-GM1 polyclonal serum (Calbiochem), the serum was diluted in PBS and incubated in streptavidin coated plates containing adsorbed antigen exposure structures for 1 h at room temperature. Bound rabbit anti GM-1 IgG was then detected with goat anti rabbit IgG-HRP (Zymed Laboratories, San Fransisco, Calif.) for 1 h and developed as described above. Between each incubation step the plate was washed three times with 0.05% Tween-20 in PBS.

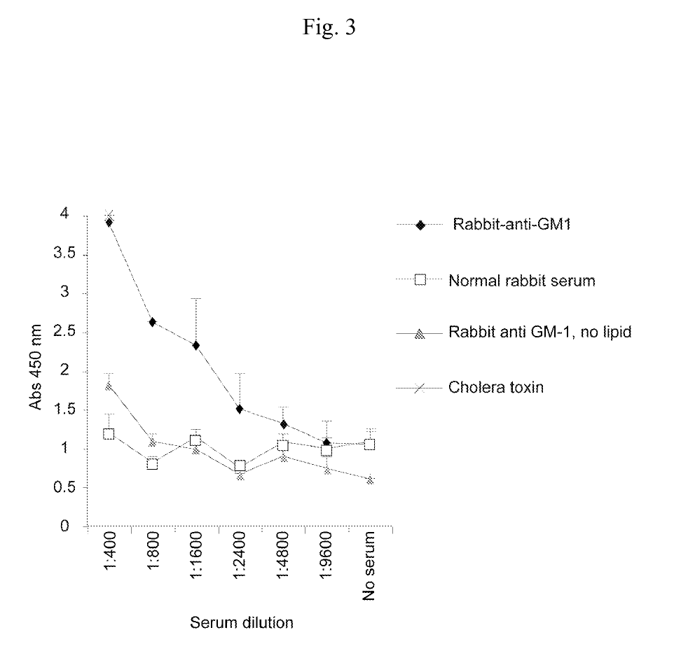

As seen from FIG. 3, a titration of the rabbit antiserum against GM-1 was obtained.

### Example 7

### Detection of Human-Anti-GM1 Antibodies in Patient Serum

Antigen presenting micelles containing GM-1 was prepared according to example 1 from GM-1, lyso-phosphatidylcholine, biotin-PE and bound to ELISA plates coated with streptavidin.

Patient serum was diluted 1:50 in PBS containing 1% BSA, and incubated for 1 h at room temperature on plates containing bound GM-1 presenting micelles. Ganglioside specific IgG or IgM was detected with alkaline phosphates conjugated anti-human IgG (IgG-ALP, Sigma-Aldrich) or IgM (IgM-ALP, Sigma-Aldrich), diluted in PBS containing 1% BSA. The plate was developed with alkaline phosphatase yellow liquid system for ELISA (Sigma-Aldrich) and absorbance 405 nm determined after 10-30 min as described above. Between each incubation step the plate was washed three times with 0.05% Tween-20 in PBS.

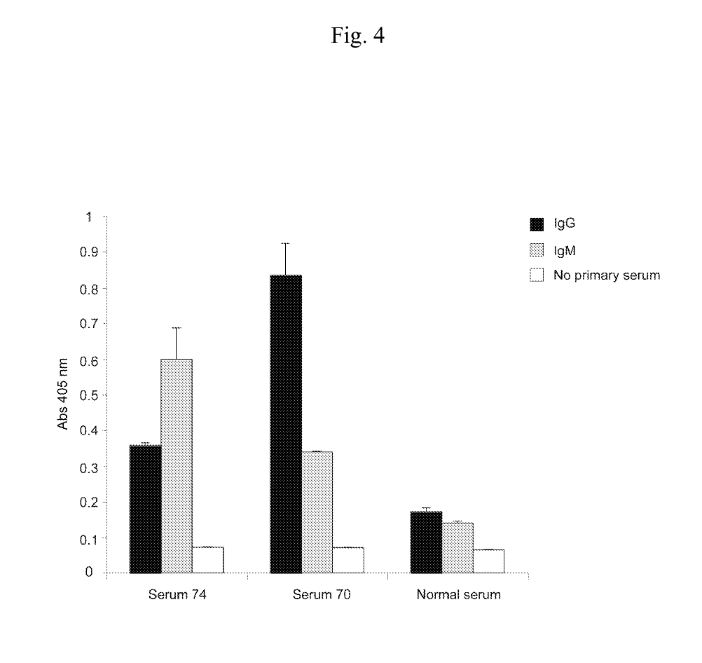

As depicted in FIG. 4, both patient sera contained higher levels of ganglioside specific IgG:s and IgM:s than the control. Furthermore the ratio between IgG and IgM differed between the patient samples.

### Example 8

### Detection of an Antigen Associated to a Phospholipid

Antigen exposing unordered aggregates were prepared by mixing biotin-PE and DNP-phosphatidylethanolamine, in chloroform. After evaporation of the organic solvent, the lipids were dispersed in PBS and bound to streptavidin coated ELISA-plates. Finally the bound antigen presenting unordered aggregates were incubated with a dilution series of an alkaline phosphatase conjugated mouse monoclonal antibody specific for DNP. The plate was developed with alkaline phosphatase yellow liquid system for ELISA (Sigma-Aldrich) and absorbance 405 nm determined as described above. Between each incubation step the plate was washed three times with 0.05% Tween-20 in PBS.

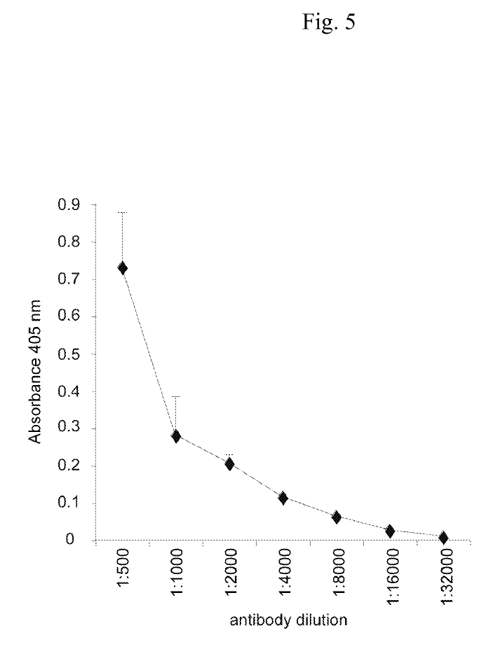

The result is depicted in FIG. 5.

### Example 9

### Detection of Antibody Binding Against Cardiolipin and Titration of the HCAL Antibody

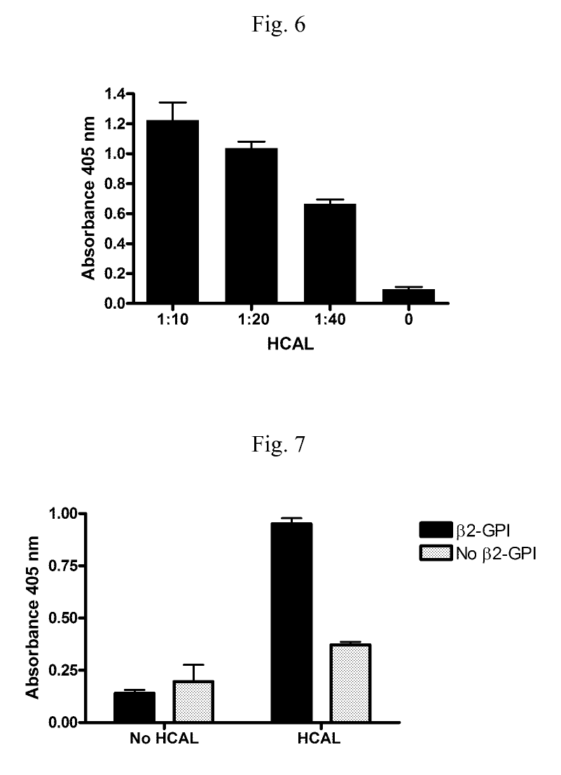

Antigen exposing unordered aggregates were prepared according to example 2 by mixing 90% cardiolipin with 10% PE-bio. The lipid aggregates were when added to 96-well streptavidin coated ELISA plates (Eurodiagnostica AB) at concentration 90 μg/ml. The plate was incubated for 1 h at room temperature. The plate was washed three times with PBS and then incubated with β2-GPI for 30 minutes. The cardiolipin-β2-GPI complex was detected by incubating with HCAL (Ichikawa, K. et al ARTHRITIS & RHEUMATISM, Vol. 42, No. 11, November 1999, pp 2461-2470), a humanized IgG antibody. To detect the HCAL antibody the plate was incubated with an alkaline phosphatase conjugated anti-human IgG antibody (Sigma-Aldrich). Between each incubation step the plate was washed three times with 0.05% Tween-20 in PBS. The result is depicted in FIG. 6. and shows titration of the HCAL antibody, which recognizes cardiolipin and a complex between cardiolipin and β2-GPI.

### Example 10

### Specificity of the HCAL Antibody and Detection of β2-GPI Associated to the Cardiolipin Aggregates

Antigen exposing lipid aggregates were prepared according to example 2 by mixing 90% cardiolipin with 10% PE-bio. The lipid aggregates were then adsorbed on a 96-well streptavidin coated ELISA plate (Eurodiagnostica AB) at concentration 90 μg/ml. The plate was incubated for 1 h at room temperature. The plate was washed three times with 0.05% Tween-20 in PBS and then incubated with or without β2-GPI for 30 minutes.

The cardiolipin-β2-GPI complex was detected by incubating with a 1:20 dilution of HCAL (Ichikawa, K. et al ARTHRITIS & RHEUMATISM, Vol. 42, No. 11, November 1999, pp 2461-2470), a humanized IgG antibody. To detect the HCAL antibody the plate was incubated with an alkaline phosphatase conjugated anti-human IgG antibody (Sigma-Aldrich). To exclude the possibility that the HCAL recognizes an epitope on the cardiolipin itself, control wells were done without β2-GPI. Between each incubation step the plate was washed three times with 0.05% Tween-20 in PBS. As seen from FIG. 7 a specific detection of HCAL against a cardiolipin-β2-GPI complex was obtained.

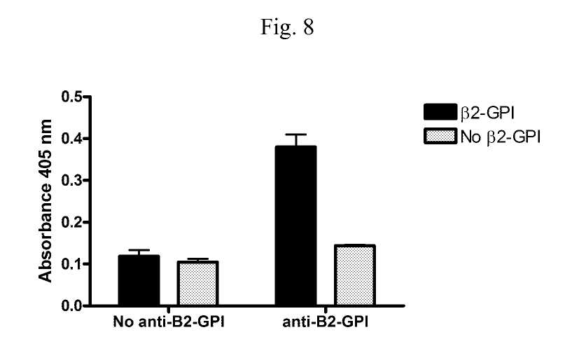

To determine the amount β2-GPI bound to cardiolipin aggregates a mouse IgG anti-β2-GPI antibody was used. This antibody was detected with an alkaline phosphatase conjugated anti-mouse IgG antibody (Sigma-Aldrich). The result is depicted in FIG. 8. and as seen from FIG. 8, β2-GPI remained associated with cardiolipin after washing with 0.05% Tween-20 in PBS.

All the antibodies were diluted in PBS with 1% BSA and incubated for 1 h at RT. The plate was developed with alkaline phosphatase yellow liquid system for ELISA (Sigma-Aldrich) and absorbance 405 nm determined after 10-30 min. Between each incubation step the plate was washed three times with 0.05% Tween-20 in PBS.

### Example 11

### Detection of Human-Anti-Cardiolipin Antibodies in Patient Serum

Antigen presenting lipid aggregates containing cardiolipin was prepared according to example 8 using cardiolipin and biotin-PE. Cardiolipin aggregates were bound to ELISA plates coated with streptavidin, washed and the adsorbed cardiolipin was expose to β2-GPI by addition β2-GPI to the wells.

The plate was washed three times with 0.05% Tween-20 in PBS and incubated with patient serum diluted 1:100 in PBS containing 0.1% BSA. After 1 h at room temperature cardiolipin-β2-GPI specific human IgG was detected using alkaline phosphates conjugated anti-human IgG (IgG-ALP, Sigma-Aldrich) diluted in PBS containing 0.1% BSA. Between each incubation step the plate was washed three times with 0.05% Tween-20 in PBS. The plate was developed with alkaline phosphatase yellow liquid system for ELISA (Sigma-Aldrich) and absorbance 405 nm determined after 10-30 min as described above.

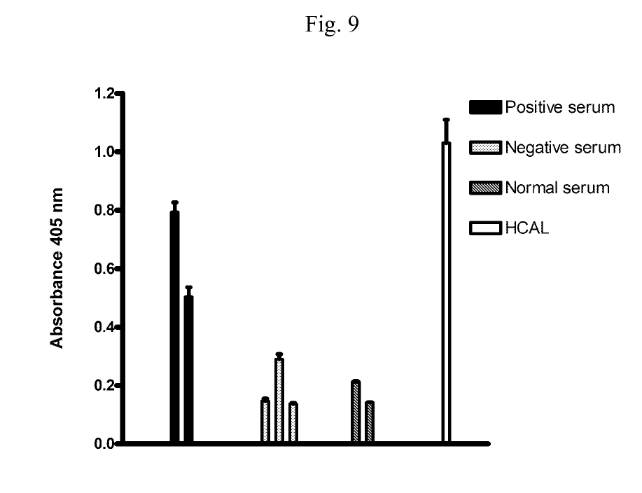

As depicted in FIG. 9, two patient sera contained higher levels of cardiolipin-β2-GPI specific IgG and three serum samples were negative. These results were all in accordance with reference data. HCAL and normal serum was run as positive and negative controls.

1.-12. (canceled)

13. An antigen exposing micelle or unordered aggregate, wherein said unordered aggregate is an amorphous type of structure having a diameter of less than 1 μm, comprising at least one carrier, at least one epitope and at least one anchoring molecule, wherein said anchoring molecule comprises at least one anchoring part, wherein said anchoring part is selected from the group consisting of biotin, biotin analogues and other biotin molecules having the ability to bind to avidin, streptavidin and derivatives thereof, wherein said anchoring part is intended to anchor the antigen exposing micelle or unordered aggregate to a surface, and wherein said micelle or said unordered aggregate comprise two or more different lipophilic structures, wherein one of said different structures is a lipophilic antigen, comprising said epitope, and another one is said anchoring molecule.

14. The micelle or unordered aggregate according to claim 13, wherein anchoring part is conjugated to an amphiphilic molecule forming unordered aggregates in aqueous solution.

15. The micelle or unordered aggregate according to claim 14, wherein the anchoring part is conjugated to phosphatidylethanolamine.

16. The micelle or unordered aggregate according to claim 15, wherein said anchoring molecule is biotinylated phosphatidylethanolamine

17. The micelle or unordered aggregate according to claim 13, wherein said carrier is lysophosphatidylcholine.

18. The micelle or unordered aggregate according to claim 13, wherein said epitope is part of a lipophilic antigen which is not said carrier or said anchoring molecule.

19. The micelle or unordered aggregate according to claim 13, wherein said epitope is part of a lipophilic antigen selected from antigens to which antibodies characterising auto-immune diseases or disorders are directed.

20. The micelle or unordered aggregate according to claim 19, wherein said lipophilic antigen is selected from the group consisting of gangliosides, cardiolipin, phospholipids and sphingolipids.

21. The micelle or unordered aggregate according to claim 13, wherein said micelle or unordered aggregate comprises from about 5 to about 20 weight % of said anchoring molecule.

22. The micelle or unordered aggregate according to claim 13, wherein said antigen exposing micelle or unordered aggregate is a micelle.

23. (canceled)

24. A kit comprising a micelle or an aggregate according to claim 13 and at least one device comprising at least one surface, wherein said anchoring part has affinity for said surface.

25. The kit according to claim 24, wherein said surface is a coated surface and wherein said coating comprises avidin or streptavidin.

26. (canceled)

27. The kit according to claim 24, which further comprises a detection reagent with affinity for an analyte, wherein said analyte has affinity for said epitope.

28. The kit according to claim 24, wherein said micelle of unordered aggregate is bound to said surface and wherein said device is an essentially dry device.

29-30. (canceled)

31. A method of detecting and/or quantifying an analyte, comprising the steps of
a) providing a micelle or unordered aggregate according to claim 1;
b) binding said micelle or unordered aggregate to a surface;
c) exposing said micelle or unordered aggregate to a sample, which sample possibly comprises said analyte, which analyte has affinity for said epitope;
d) washing said bound micelle or unordered aggregate with a solution comprising a detergent; and
e) detecting and/or quantifying said analyte.

32. The method according to claim 31, wherein said sample is plasma or serum form a mammal.

33. The method according to claim 16, wherein said analyte is an auto-antibody.
